(19) United States
(12) Patent Application Publication
(10) Pub. No.: US 2006/0057206 A1
(43) Pub. Date: Mar. 16, 2006

(54) Controlled release nanoparticle active agent formulation dosage forms and methods

(76) Inventors: Patrick S.L. Wong, Burlingame, CA (US); Liang-Chang Dong, Sunnyvale, CA (US); Ruiping Zhao, San Jose, CA (US); Crystal Pollock-Dove, Mountain View, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/198,717

(22) Filed: Aug. 4, 2005

Related U.S. Application Data
(60) Provisional application No. 60/603,134, filed on Aug. 19, 2004.

(51) Int. Cl. A61K 9/24

(52) U.S. Cl. 424/473

(57) ABSTRACT

Controlled release of self-dispersing nanoparticle active agent formulations is provided by dispersing porous particles into which have been sorbed a self-dispersing nanoparticle active agent formulation in osmotic, push-layer dosage forms. The dosage forms may provide for continuous or pulsatile delivery of active agents.

### CROSS-REFERENCE TO RELATED APPLICATION

This application claims benefit, under 35 U.S.C 119(e), of U.S. Ser. No. 60/603,134, filed Aug. 19, 2004, which is incorporated herein by reference.

### FIELD OF THE INVENTION

This invention pertains to the controlled delivery of pharmaceutical agents and dosage forms therefor. In particular, the invention is directed to improved methods, dosage forms and devices for the controlled delivery of liquid active agent formulations to an environment of use.

### BACKGROUND OF THE INVENTION

The present inventors have previously taught and disclosed methods and devices, such as described in U.S. Pat. No. 6,342,249, incorporated herein by reference, for the controlled release of liquid, active agent formulations. The liquid, active agent formulations were loaded into porous particles that served as carriers for the liquid active agent formulations. The porous particles, loaded with liquid active agent formulations, could be formulated into osmotic, push-layer dosage forms. For certain drugs, the methods and devices taught in U.S. Pat. No. 6,342,249 do not provide optimal results and, in fact, present undesirable limitations, particularly in the aspect of dosage loading.

In past practice, administration of liquid active agent formulations was often preferred over solid active agent formulations in order to facilitate absorption of the active agent and obtain a beneficial effect for the intended use in the shortest possible time after the formulation is exposed to the environment of use. Examples of prior art devices to deliver liquid active agent formulations are soft gelatin capsules that contain a liquid active agent formulation or liquid formulations of the active agent that are bottled and dispensed in measured dosage amounts by the spoonful, or the like. Those systems are not generally amenable to controlled delivery of the active agent over time. While it is desired to have the active agent exhibit its effect as soon as it is released to the environment of use, it also often is desirable to have controlled release of the active agent to the environment of use over time. Such controlled release may be sustained delivery over time, such as zero order, or patterned delivery, such as pulsatile for example. Prior art systems have not generally been suitable for such delivery.

Various devices and methods have been described for the continuous delivery of active agents over time. Typically, such prior art systems have been used to deliver active agents initially in the dry state prior to administration. For example, U.S. Pat. Nos. 4,892,778 and 4,940,465, which are incorporated herein by reference, describe dispensers for delivering a beneficial agent to an environment of use that include a semipermeable wall defining a compartment containing a layer of expandable material that pushes a drug layer out of the compartment formed by the wall. The exit orifice in the device is substantially the same diameter as the inner diameter of the compartment formed by the wall.

U.S. Pat. No. 4,915,949, which is incorporated herein by reference, describes a dispenser for delivering a beneficial agent to an environment of use that includes a semipermeable wall containing a layer of expandable material that pushes a drug layer out of the compartment formed by the wall. The drug layer contains discrete tiny pills dispersed in a carrier. The exit orifice in the device is substantially the same diameter as the inner diameter of the compartment formed by the wall.

U.S. Pat. No. 5,126,142, which is incorporated herein by reference, describes a device for delivering an ionophore to livestock that includes a semipermeable housing in which a composition containing the ionophore and a carrier and an expandable hydrophilic layer is located, along with an additional element that imparts sufficient density to the device to retain it in the rumen-reticular sac of a ruminant animal. The ionophore and carrier are present in a dry state during storage and the composition changes to a dispensable, fluid-like state when it is in contact with the fluid environment of use. A number of different exit arrangements are described, including a plurality of holes in the end of the device and a single exit of varying diameter to control the amount of drug released per unit time due to diffusion and osmotic pumping.

It is often preferable that a large orifice, from about 50%-100% of the inner diameter of the drug compartment, be provided in the dispensing device containing the active agent and a bioerodible or degradable active agent carrier. When exposed to the environment of use, drug is released from the drug layer by erosion and diffusion. In those prior art instances where the drug is present in the solid state, the realization of the beneficial effect is delayed until the drug is dissolved in the fluids of the environment of use and absorbed by the tissues or mucosal environment of the gastrointestinal tract. For drugs that are poorly soluble in gastric or intestinal fluids, these delays found in the prior art are not preferred.

Devices in which the drug composition initially is dry but in the environment of use is delivered as a slurry, suspension or solution from a small exit orifice by the action of an expandable layer are described in U.S. Pat. Nos. 5,660,861, 5,633,011; 5,190,765; 5,252,338; 5,620,705; 4,931,285; 5,006,346; 5,024,842; and 5,160,743. Typical devices include an expandable push layer and a drug layer surrounded by a semipermeable membrane.

When the active agent is insoluble or poorly soluble, prior art systems may not provide rapid delivery of active agent or concentration gradients at the site of absorption that facilitate absorption through the gastrointestinal tract. Various approaches have been put forth to address such problems, including the use of water-soluble salts, polymorphic forms, powdered solutions, molecular complexes, micronization, eutectics, and solid solutions. An example of the use of a powdered solution is described by Sheth, et al., in “Use of Powdered Solutions to Improve the Dissolution Rate of Polythiazide Tablets,” Drug Development and Industrial Pharmacy, 16(5), 769-777 (1990). References to certain of the other approaches are cited therein. Additional examples of powdered solutions are described in U.S. Pat. No. 5,800,834. The patent describes methodology for calculating the amount of liquid that may be optimally sorbed into materials to prevent the drug solution from being exuded from the granular composition during compression.

U.S. Pat. No. 5,486,365, which is incorporated herein by reference, describes a spheronized material formed from a scale-like calcium hydrogen phosphate particulate material having a high specific surface area, good compressibility and low friability. That patent indicates that the material has the characteristic of high liquid absorption. However, the patent does not suggest that the material may be used as a carrier for delivery of a liquid medicament formulation to the environment of use. Instead, the patent describes the formation of a dried formulation, such as formed by spray drying. The patent describes the use of a suspension containing medicines and binders during the spray-drying granulation process to form a spherical particle containing the medicine. As an example, ascorbic acid in an amount equivalent to 10% of the scale-like calcium hydrogen phosphate was dissolved into a slurry of 20 weight percent of calcium hydrogen phosphate in water, and the resulting slurry was spray dried to form dried, spherical calcium hydrogen phosphate containing ascorbic acid. That material was then tableted under loads of 500-2000 kg/cm².

### SUMMARY OF THE INVENTION

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles; the porous particles having a mean particle size of ranging from about 50 to about 150 microns and being formed by spray drying a scale-like calcium hydrogen phosphate with a specific surface area of about 20 m²/g to about 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a bulk specific volume of 1.5 ml/g-5 ml/g, a BET specific area of 20 m²/g-60 m²/g, a water absorption capacity of at least 0.7 ml/g, and a mean particle size of at least 70 micrometers.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation.

In an aspect, the invention relates to method of facilitating the release of an active agent from a dosage form comprising: sorbing a self-dispersing nanoparticle active agent formulation of the active agent into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0; and dispersing the particles throughout a bioerodible carrier.

In an aspect, the invention relates to a composition comprising a self-dispersing nanoparticle active agent formulation of the active agent sorbed into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0, and dispersed throughout a bioerodible carrier, the particles being released in the environment of use over a prolonged period of time.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being magnesium aluminometasilicate.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being magnesium aluminometasilicate represented by the general formula:
Al₂O₃MgO.2SiO₂.n H₂O
wherein n satisfies the relationship 0≦n≦10.

In an aspect, the invention relates to a dosage form for an active agent comprising: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in and/or onto porous particles, the porous particles being magnesium aluminometasilicate represented by the general formula
Al₂O₃MgO.2SiO₂.nH₂O
wherein n satisfies the relationship 0≦n≦10 and having a specific surface area of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g.

In an aspect, the invention relates to a composition of nanoparticles of an active agent suspended in a liquid carrier and sorbed into porous particle carriers.

19. A dosage form comprising a self-dispersing nanoparticle formulation loaded into one or more porous carriers and wherein the nanoparticles have a mean particle size less than 2000 nm.

### BRIEF DESCRIPTION OF THE FIGURES

FIG. 1 illustrates a porous particle containing a self-dispersing active agent formulation according to the present invention;

FIG. 2 illustrates a composition comprising a plurality of particles containing a self-dispersing nanoparticle active agent formulation as illustrated in FIG. 1 dispersed in a carrier and suitable for use in dosage forms of the invention;

FIG. 3 illustrates a dosage form of this invention adapted for zero order release of active agent;

FIG. 4 illustrates a dosage form of this invention adapted to deliver a delayed pulse of the active agent;

FIG. 5 illustrates the release profile (cumulative release as a function of time) of the active agent megestrol acetate from a representative dosage form of the present invention as described in Example 8.

FIG. 6 illustrates the bioavailability of megestrol acetate from a representative dosage form of the present invention as described in Example 9 and as compared to the bioavailability of the commercial product Megace (® B-M).

### DETAILED DESCRIPTION OF THE INVENTION

The present invention is best understood by reference to the following definitions, the drawings and exemplary disclosure provided herein.

Definitions

By “active agent”, “drug”, or “compound”, which are used interchangeably herein, is meant an agent, drug, compound, composition of matter or mixture thereof which provides some physiological, psychological, biological, or pharmacological, and often beneficial, effect when in the environment of use.

By “uniform rate of release” or “uniform release rate” is meant a rate of release of the active agent from a dosage form that does not vary positively or negatively by more than 30% from the mean rate of release of the active agent over a prolonged period of time, as determined in a USP Type 7 Interval Release Apparatus. Preferred uniform rates of release will vary by not more than 25% (positively or negatively) from the mean rate of release determined over a prolonged period of time.

By “prolonged period of time” or “prolonged period” is meant a continuous period of time of 4 hours or more, more typically 6 hours or more.

By “dosage form” is meant a pharmaceutical composition or device comprising an active pharmaceutical agent, the composition or device optionally containing inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like, that are used to manufacture and deliver active pharmaceutical agents.

By “pharmaceutically-acceptable acid addition salt” or “pharmaceutically-acceptable salt”, which are used interchangeably herein, are meant those salts in which the anion does not contribute significantly to the toxicity or pharmacological activity of the salt, and, as such, they are the pharmacological equivalents of the bases of the compounds to which they refer. Examples of pharmaceutically acceptable acids that are useful for the purposes of salt formation include but are not limited to hydrochloric, hydrobromic, hydroiodic, citric, acetic, benzoic, mandelic, fumaric, succinic, phosphoric, nitric, mucic, isethionic, palmitic, and others.

By “sustained release” is meant continuous release of active agent to an environment of use over a prolonged period.

By “pulsatile release” is meant release of an active agent to an environment of use for one or more discrete periods of time preceded or followed by (i) at least one discrete period of time in which the active agent is not released, or (ii) at least one period of time in which another, different active agent is released. Pulsatile release is meant to include delayed release of active agent following administration of the dosage form and release in which one or more pulses of active agent are released over a period of time.

By “steady state” is meant the condition in which the amount of drug present in the blood plasma of a subject does not vary significantly over a prolonged period of time.

By “release rate assay” is meant a standardized assay for the determination of a compound using a USP Type 7 interval release apparatus substantially in accordance with the description of the assay contained herein. It is understood that reagents of equivalent grade may be substituted in the assay in accordance with generally-accepted procedures. Also, different fluids such as artificial gastric fluid or artificial intestinal fluid may be used to evaluate release characteristics in environments characterized by different pH values.

By “liquid, active agent formulation” is meant that the active agent is present in a composition that is miscible with or dispersible in the fluids of the environment of use, or is able to flow or diffuse from the pores of the particles into the environment of use. The formulation may be neat, liquid active agent, or a solution, suspension, slurry, emulsion, self-emulsifying composition, colloidal dispersion or other flowable composition in which the active agent is present.

The active agent may be accompanied by a suspension agent, antioxidant, emulsion former, protecting agent, permeation enhancer and the like. The amount of an active agent in a dosage form generally is about 0.05 ng to 5 g or more, with individual dosage forms comprising, for example, 25 ng, 1 mg, 5 mg, 10 mg, 25 mg, 100 mg, 250 mg, 500 mg, 750 mg, 1.0 g, 1.2 g, and the like, of active agent. The system typically can be administered once, twice or thrice daily for pharmaceutical applications, or more or less as required by the particular application. In agricultural applications, systems typically will be applied at longer intervals, such as weekly, monthly, seasonally or the like.

By “self-dispersing nanoparticle active agent formulation” is meant a liquid active agent formulation which comprises nanoparticles of active agent and which can disperse in an aqueous medium without vigorous agitation. Some of the active agent in a self-dispersing nanoparticle active agent formulation may also be dissolved in the liquid active agent formulation. The formulation serves to disperse in the gastrointestinal environment and can provide emulsion vehicles or emulsion bodies that distribute the nanoparticles in the gastrointestinal environment and also facilitate enhanced dissolution of the active agent in the gastrointestinal environment.

By “nanoparticle” of drug is meant a drug particle having a mean particle size smaller than 2000 nm, more preferably 30 to 1500 nm, more preferably 100 to 1000 nm, more preferably 200 to 600 nm. Additionally, the particles may preferably have a mean particle size of less than 1500 nm, more preferably less than 1000 nm, and more preferably less than 600 nm.

By “porous carrier” is meant a plurality of porous particles or porous particulates of a homogenous or heterogenous composition.

By “formulation loaded into the porous carrier” or “formulation loaded into the porous particles” is meant that the formulations are sorbed into, onto or otherwise mixed with the porous particles of the porous particle carrier.

It has been found that various beneficial effects are gained by the use of drug nanoparticles as active agents in a self-dispersing nanoparticle active agent formulation loaded into and/or onto porous particles. This is particularly true for drugs that exhibit low solubility in the gastrointestinal environment such as Class II and Class IV drugs as defined by the U.S. FDA Biopharmaceutical Classification System. Prior to the present invention, it has been difficult to provide a combination of high dosage loading and high dissolution characteristics in a dosage form for such low solubility drugs. As an aspect of the present invention, the self-dispersing carrier provides significantly enhanced solubility for the drug once the drug form releases its contents in the gastrointestinal system. Benefits are derived that result directly from the characteristics of the nanoparticles. Additional benefits arise from the combination of the nanoparticles, porous carrier and the self-dispersing carrier.

In some embodiments of the present invention the self-dispersing nanoparticle formulation is in the form of emulsions or self-emulsifying compositions as defined herein. Due to the increased solubility of the drug provided by the self-emulsifying composition, creation of relatively higher concentrations of dissolved drug in the gastrointestinal tract are achieved. Moreover, because the emulsion works to solubilize the drug in the gastrointestinal environment as the already dissolved drug material is absorbed by the body, the self-emulsifying suspension works to maintain a higher concentration of dissolved drug in the gastrointestinal tract over a longer period of time than would be possible if the formulation simply included an amount of the dissolved drug. This, then, leads to preferred faster and greater absorption of the drug.

In certain preferred embodiments, the self-dispersing nanoparticle formulation is one in which the formulation, when released from a dosage form in the gastrointestinal tract, can disperse in the aqueous media of the gastrointestinal tract without vigorous agitation, or in other words, can disperse in the aqueous media of the gastrointestinal tract by effect of the motility of the gastrointestinal tract.

Some of the benefits of the present invention arise from characteristics of the nanoparticles themselves. Nanoparticles of a drug dissolve more quickly than larger sized particles of the same drug. One reason is that, since geometrically an equal weight of nanoparticles has a greater surface area than does an equal weight of larger particles of the same drug, a nanoparticle form of a drug has a greater surface area available for dissolution of the drug from the drug particles or crystals than does an equal weight of the drug in a form composed of larger sized particles. Additionally, nanoparticles inherently have a more irregular surface area and crystal structure than do larger more regular drug crystals. Since dissolution from the irregular surface crystal structure of nanoparticles occurs more readily than from a regular crystal surface and structure of larger sized particles, nanoparticles dissolve more readily than do larger particles of the same drug.

If nanoparticles are simply packed into a drug form without the other aspects of the present invention, the drug particles or crystals tend to combine or agglomerate. The resultant larger drug particles of the drug form undesirably dissolve more slowly in the gastrointestinal tract than do non-agglomerated nanoparticles of the drug.

By mixing the drug nanoparticles into a self-dispersing carrier and then loading the resulting self-dispersing nanoparticle formulation into porous particle carriers, undesired growth or agglomeration of drug particles is inhibited. When drug nanoparticles are mixed into a self-dispersing carrier without then loading the mixture into porous particle carriers, it is typical that the nanoparticles, or at least usually the larger nanoparticles, will grow by the phenomenon of Oswald ripening. However, when such a mixture is loaded into porous particle carriers the porous particles tend to provide, in many of the preferred formulations, a physical separation between the nanoparticles (and, as explained below, between portions of the liquid carrier) and will minimize or eliminate Oswald ripening growth of a substantial portion of the nanoparticles. It should be understood that the porous particles, by capillary and other actions, absorb the bulk of the liquid carrier and thus provide a physical separation between the nanoparticles and also virtually eliminate liquid communication between the nanoparticles. This eliminates or largely prevents Oswald ripening induced growth of the nanoparticles. This presents obvious advantages over systems in which the nanoparticles are packed together in a dosage form and then tend to agglomerate. Clearly, these aspects of the present invention present beneficial advantages over systems in which nanoparticles are provided in suspension (without loading into porous carriers) wherein the nanoparticles of such systems frequently grow, agglomerate or combine during storage in the suspension formulation. This growth or agglomeration diminishes the solubility of the drug and effectiveness of the drug forms in which the drug is embodied. Additionally, such suspensions of nanoparticles cannot be handled with dosage form manufacturing equipment designed to process dry constituents of dosage forms, while self-dispersing nanoparticle formulations sorbed into porous particle carriers according to the present invention can be processed by such equipment.

According to various aspects or embodiments of the present invention, the self-dispersing nanoparticle active agent formulation can be sorbed into the pores of the porous particle carrier. Additionally, nanoparticles of the formulation can adhere to the outside of the porous particle for reasons such as the wetness of the surface of the porous particle effected by the self-dispersing formulation.

The present invention achieves the combined objectives of a high drug loading while maintaining and without compromising high dissolution characteristics.

Nanoparticles used in the present invention preferably have a mean particle size less than 2000 nm, more preferably they range from 20 to 2000 nm, more preferably 30 to 1500 nm, even more preferably 100 to 1000 nm, more preferably 200 to 600 nm. Additionally, the particles may preferably have a mean particle size of less than 1500 nm, more preferably less than 1000 nm, and more preferably less than 600 nm.

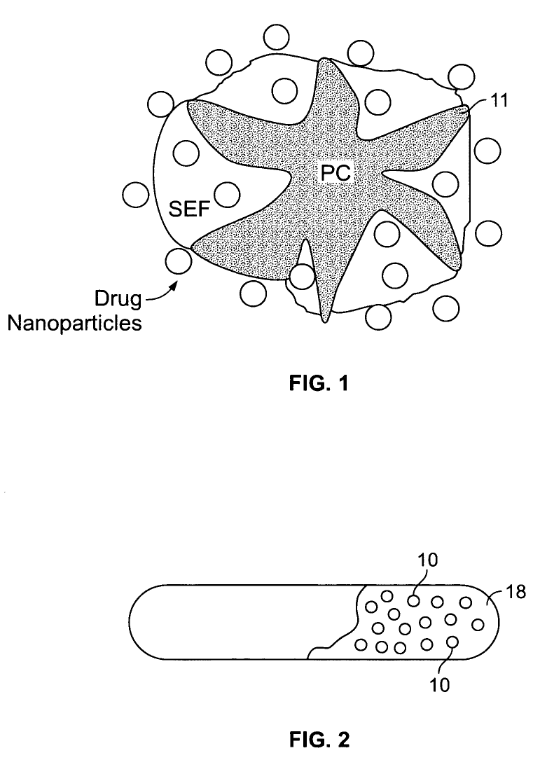

FIG. 1 illustrates a porous particle 10 having a material mass 11 that defines a plurality of pores 12 and which has been loaded with a self-dispersing nanoparticle formulation 14 comprising a self-dispersing liquid carrier and active agent nanoparticles 16. Within pores 12 is sorbed the self-dispersing formulation 14. Nanoparticles 16 are not only contained in the pores 12 but also can adhere to the outside of the porous particle 10 due to factors such as potential wetness of the surface of porous particle 10 effected by the self-dispersing formulation 14. Pores 14 extend from the external surface of the particle and into the interior. Pores are open on the surface to permit the self-dispersing nanoparticle active agent formulation to be sorbed into the particles by conventional mixing techniques such as wet granulation, spraying of the self-dispersing nanoparticle active agent formulation onto a fluidized bed of the particles, or the like. Additionally, according to embodiments of the present invention, some percentage of the drug may be dissolved in the liquid carrier.

One of the most suitable devices for the controlled release of self-dispersing nanoparticle active agent formulations in accordance with this invention is that having a semipermeable wall defining a compartment, an expandable push layer and a drug layer in the compartment, and an exit orifice formed in the dosage form to permit the drug layer to be dispensed. Within the drug layer is a carrier in which is dispersed a plurality of porous particles in which the self-dispersing nanoparticle active agent has been sorbed. As the push layer expands, the carrier comprising the drug layer will be forced from the dosage form substantially in the dry state where it will erode and release the porous particles containing the self-dispersing nanoparticle active agent formulation. After release, the self-dispersing components tend to disperse the nanoparticles in the gastrointestinal environment. The self-emulsifying characteristics of the self-dispersing formulation tend to distribute the nanoparticles and facilitate their dissolution in the gastrointestinal environment. se the active agent formulation.

When manufacturing such dosage forms, a common practice is to fabricate a compressed tablet comprising the drug layer and the push layer. Typically, the drug layer composition, conveniently in granulated or powdered form, is compressed in a die cavity of a vertical tabletting press. Then the push layer composition, also conveniently in granular or powdered form, is placed in the die cavity above the drug layer and compressed as well to form a bilayer tablet. During the compression or compacting step of the drug layer, the porous particles should be sufficiently resistant to the compressive forces so as not to be crushed or pulverized to any significant extent and prematurely release the self-dispersing nanoparticle active agent formulation from the porous particles.

Materials useful for sorbing the self-dispersing nanoparticle active agent formulations are porous particulates that are characterized by high compressibility or tensile strength to withstand compacting forces applied during compacting steps and minimize exudation of self-dispersing nanoparticleself-dispersing nanoparticle active agent formulation from the pores; particle flow characteristics that allow for the porous particles to be directly compacted without the use of a binder or with minimal use of a binder; low friability so as to preclude or minimize exudation of the liquid and facilitate tablet cohesion, active agent formulation from the particles during compacting steps; and high porosity so as to absorb an adequate of amount of a self-dispersing nanoparticle active agent formulation to provide an effective amount of active agent in a dosage form. The particles should be adapted to absorb an amount of self-dispersing nanoparticles active agent formulation such that a therapeutically effective amount of the active agent may be delivered in a unitary dosage form that is of a size that can be conveniently swallowed by a subject and, preferably provided in four or fewer tablets or capsules for ingestion at the same time. The porosity of the particles may be such that at least 5% and up to 70%, more often 20-70%, preferably 30-60%, and more preferably 40-60%, by weight of the self-dispersing nanoparticle active agent formulation, based on weight of the particles may be sorbed into the pores of the particles, while the particles exhibit sufficient strength at such degree of active agent loading so as not to significantly be crushed or pulverized by compacting forces to which the particles will be subjected during manufacturing operations. More typically, the self-dispersing nanoparticle active agent formulation may comprise 30-40% of the weight of the porous particles when the particles are crystalline, such as calcium hydrogen phosphate, but that percentage may be greater, e.g., up to 60-70% or more when more amorphous materials, such as magnesium aluminometasilicates, are used. Blends of crystalline and amorphous material may be utilized. At high loadings, it may be advantageous to use blends of calcium hydrogen phosphate particles and amorphous magnesium aluminometasilicate powders.

Preferred materials are those having a strength to resist compression forces of greater than 1500 kg/cm² without substantial exudation of the self-dispersing nanoparticle active agent formulation, and most preferably without the tablet hardness plateauing.

A particularly suitable porous particle is exemplified by the particular form of calcium hydrogen phosphate described in U.S. Pat. No. 5,486,365, which is incorporated herein by reference. As described therein, calcium hydrogen phosphate is prepared by a process yielding a scale-like calcium hydrogen phosphate that can be represented by the formula CaHPO₄.mH₂O wherein m satisfies the expression 0≦m≦0.5. Useful calcium hydrogen phosphate materials may include those of the formula CaHPO₄.mH₂O wherein m satisfies the expression 0≦m≦2.0. The scale-like calcium hydrogen phosphate produced has characteristic physical properties that make it particularly suitable for use in the present invention. The scale-like material provides high specific surface area, high specific volume, high capacity for water and oil absorption, and the ability to readily form into spheres upon spray drying. The spherical particulates have excellent flow properties and permit direct compaction into tablets without binders and without significant crushing or pulverizing of the particles during the compaction step.

The scale-like calcium hydrogen phosphate particles generally have a BET specific surface area of at least 20 m²/g, typically 20 m²/g -60 m²/g, a specific volume of at least 1.5 ml/g, typically 2-5 ml/g or more, and an oil and water absorption capacity of at least 0.7 ml/g, typically 0.8-1.5 ml/g. When formed into spheres the spherical particulates may have a mean particle size a mean particle size of 50 microns or greater, usually about 50-150 microns, and often about 60-120 microns. The particle size distribution may be 100% through 40 mesh, 50%-100% through 100 mesh, and 20%-60% through 200 mesh. The bulk density may be from about 0.4 g/ml-0.6 g/ml.

A most preferred form of calcium hydrogen phosphate is that sold under the trademark FujiCalin® by Fuji Chemical Industries (U.S.A.) Inc., Robbinsville, N.J., in types SG and S. Typical parameters for that material include a mean particle size of 500-150 microns, a mean pore size on the order of 70 Angstroms, a specific volume of about 2 ml/g, a BET specific surface area of about 30-40 m²/g, and an oil and water absorption capacity of about 0.7 ml/g. Type SG typically will have a mean particle size of about 113 microns, and a particle size distribution of 100% through 40 mesh, 60% through 100 mesh and 20 through 200 mesh. Type S typically will have a mean particle size of about 68 microns, and a particle size distribution of 100% through 40 mesh, 90% through 100 mesh and 60% through 200 mesh. Mixtures of the two types may be conveniently employed to provide particulates having physical characteristics that are suitable for various applications, as may be determined by those skilled in the art of pharmaceutical formulation, tableting and manufacturing.

The calcium hydrogen phosphate has low friability, demonstrating a tensile strength of up to about 130 kg/cm² when subjected to compressive forces of up to 3000 kg/cm². The hardness of the tableted material tends not to plateau at compression forces to that limit, while materials such as microcrystalline cellulose (Avicel PH 301), lactose, DI-TAB and Kyowa GS tend to plateau at or about 700-1500 Kg/cm². The angle of repose for the preferred materials typically is on the order of 32-35 degrees.

Another material that may be utilized is that formed of magnesium aluminometasilicate which may be represented by the general formula
Al₂O₃MgO.2SiO₂.nH₂O
wherein n satisfies the relationship 0≦n≦10. Commercially available magnesium aluminometasilicates are sold as Grades S₁, SG₁, UFL₂, US₂, FH₁, FH₂, FL₁, FL₂, S₂, SG₂, NFL₂N, and NS₂N, under the trademark Neusilin™ by Fuji Chemical Industries (U.S.A.) Inc., Robbinsville, N.J. Especially preferred grades are S₁, SG₁, US₂ and UFL₂, with US₂ presently being most preferred. Those materials which are amorphous typically have a specific surface area (arca) of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g.

Other absorptive materials may be substituted for the foregoing or blended therewith, such as for example, powders of microcrystalline cellulose sold under the tradenames Avicel (FMC Corporation) and Elcema (Degussa); porous sodium carboxymethyl cellulose crosslinked sold as Ac-Di-Sol (FMC Corporation); porous soy bean hull fiber sold under the tradename FI-1 Soy Fiber (Fibred Group); and porous agglomerated silicon dioxide, sold under the tradenames Cab-O-Sil (Cabot) and Aerosil (Degussa).

The self-dispersing nanoparticle active agent formulation may be in any form that can be dispensed from the porous particles as the drug layer disintegrates in the environment of use. Optionally other dosage-forming ingredients, such as an anti-oxidant, a suspending agent, a surface active agent, and the like may be present in the self-dispersing nanoparticle active agent formulation. The self-dispersing nanoparticle active agent formulation will be released in a form most suitable to provide active agent to the site of delivery in a state in which it may be rapidly dissolved and absorbed in the environment of use to provide its beneficial action with minimum delay once delivered to the absorption site.

It often is desirable to provide the dosage form with a flow-promoting layer or lubricant that facilitates complete release of the drug layer from the compartment formed by the semipermeable wall since the formed bilayer tablet may be formed with surface irregularities that impede the release of the drug layer from the dosage form and sometimes results in incomplete release of the drug layer.

Dosage forms of this invention release effective amounts of active agent to the patient over a prolonged period of time and often provide the opportunity for less frequent dosing, including once-a-day dosing, than previously required for immediate release compositions. The dosage forms of some embodiments of this invention comprise a composition containing a self-dispersing nanoparticle active agent formulation contained in porous particles dispersed in a bioerodible carrier.

Active agents include, inter allia, foods, food supplements, nutrients, drugs, antiacids, vitamins, microorganism attenuators and other agents that provide a benefit in the environment of use. Active agents include any physiologically or pharmacologically active substance that produces a localized or systemic effect or effects in animals, including warm blooded mammals, humans and primates; domestic household or farm animals such as cats, dogs, sheep, goats, cattle, horses and pigs; laboratory animals such as mice, rats and guinea pigs; zoo and wild animals; and the like. Active agents that can be delivered include inorganic and organic compounds, including, without limitation, active agents which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system.

Suitable active agents may be selected from, for example, proteins, enzymes, enzyme inhibitors, hormones, polynucleotides, nucleoproteins, polysaccharides, glycoproteins, lipoproteins, polypeptides, steroids, hypnotics and sedatives, psychic energizers, tranquilizers, anticonvulsants, antidepressants, muscle relaxants, antiparkinson agents, analgesics, anti-inflammatories, antihystamines, local anesthetics, muscle contractants, antimicrobials, antimalarials, antivirals, antibiotics, antiobesity agents, hormonal agents including contraceptives, sympathomimetics, polypeptides and proteins capable of eliciting physiological effects, diuretics, lipid regulating agents, antiandrogenic agents, antiparasitics, neoplastics, antineoplastics, antihyperglycemics, hypoglycemics, nutritional agents and supplements, growth supplements, fats, ophthalmics, antienteritis agents, electrolytes and diagnostic agents.

Examples of particular active agents useful in this invention include prochlorperazine edisylate, ferrous sulfate, albuterol, aminocaproic acid, mecamylamine hydrochloride, procainamide hydrochloride, amphetamine sulfate, methamphetamine hydrochloride, benzphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperazine maleate, anisindione, diphenadione erythrityl tetranitrate, digoxin, isoflurophate, acetazolamide, nifedipine, methazolamide, bendroflumethiazide, chlorpropamide, glipizide, glyburide, gliclazide, tobutamide, chlorproamide, tolazamide, acetohexamide, mefformin, troglitazone, orlistat, bupropion, nefazodone, tolazamide, chlormadinone acetate, phenaglycodol, allopurinol, aluminum aspirin, methotrexate, acetyl sulfisoxazole, hydrocortisone, hydrocorticosterone acetate, cortisone acetate, dexamethasone and its derivatives such as betamethasone, triamcinolone, methyltestosterone, 17-β-estradiol, ethinyl estradiol, ethinyl estradiol 3-methyl ether, prednisolone, 17-β-hydroxyprogesterone acetate, 19-nor-progesterone, norgestrel, norethindrone, norethisterone, norethiederone, progesterone, norgesterone, norethynodrel, terfandine, fexofenadine, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, selegiline, chlorpromazine, methyldopa, dihydroxyphenylalanine, calcium gluconate, ketoprofen, ibuprofen, cephalexin, erythromycin, haloperidol, zomepirac, ferrous lactate, vincamine, phenoxybenzamine, diltiazem, milrinone, captropril, mandol, quanbenz, hydrochlorothiazide, ranitidine, flurbiprofen, fenbufen, fluprofen, tolmetin, alclofenac, mefenamic, flufenamic, difuninal, nimodipine, nitrendipine, nisoldipine, nicardipine, felodipine, lidoflazine, tiapamil, gallopamil, amlodipine, mioflazine, lisinopril, enalapril, captopril, ramipril, enalaprilat, famotidine, nizatidine, sucralfate, etintidine, tetratolol, minoxidil, chlordiazepoxide, diazepam, amitriptyline, and imipramine, and pharmaceutical salts of these active agents. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatropin, oxytocin, vasopressin, prolactin, somatostatin, lypressin, pancreozymin, luteinizing hormone, LHRH, interferons, interleukins, growth hormones such as human growth hormone, bovine growth hormone and porcine growth hormone, fertility inhibitors such as the prostaglandins, fertility promoters, growth factors, and human pancreas hormone releasing factor.

The present invention has particular utility in the delivery of self-dispersing nanoparticle active agent formulations that are in the form of emulsions or self-emulsifying compositions. The term emulsion as used in this specification denotes a two-phase system in which one phase is finely dispersed in the other phase. The term emulsifier, as used by this invention, denotes an agent that can reduce and/or eliminate the surface and the interfacial tension in a two-phase system. The emulsifier agent, as used herein, denotes an agent possessing both hydrophilic and lipophilic groups in the emulsifier agent. The term microemulsion, as used herein, denotes a multicomponent system that exhibits a homogenous single phase in which quantities of a drug can be solubilized. Typically, a microemulsion can be recognized and distinguished from ordinary emulsions in that the microemulsion is more stable and usually substantially transparent. The term solution, as used herein, indicates a chemically and physically homogenous mixture of two or more substances.

The emulsion formulations of active agent generally comprise 0.5 wt % to 99 wt % of a surfactant. The surfactant functions to prevent aggregation, reduce interfacial tension between constituents, enhance the free-flow of constituents, and lessen the incidence of constituent retention in the dosage form. The therapeutic emulsion formulations useful in this invention may comprise a surfactant that imparts emulsification comprising a member selected from the group consisting of polyoxyethylenated castor oil comprising 9 moles of ethylene oxide, polyoxyethylenated castor oil comprising 15 moles of ethylene oxide, polyoxyethylene castor oil comprising 20 moles of ethylene oxide, polyoxyethylenated castor oil comprising 25 moles of ethylene oxide, polyoxyethylenated castor oil comprising 40 moles of ethylene oxide, polyoxylenated castor oil comprising 52 moles of ethylene oxide, polyoxyethylenated sorbitan monopalmitate comprising 20 moles of ethylene oxide, polyoxyethylenated sorbitan monolaurate comprising 20 moles of ethylene oxide, polyoxyethylenated sorbitan monooleate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylenated stearic acid comprising 8 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. The surfactants are available from Atlas Chemical Industries, Wilmington, Del.; Drew Chemical Corp., Boonton, N.J.; and GAF Corp., New York, N.Y.

Typically, an active agent emulsified formulation useful in the invention initially comprises an oil phase. The oil phase of the emulsion comprises any pharmaceutically acceptable oil which is not miscible with water. The oil can be an edible liquid such as a non-polar ester of an unsaturated fatty acid, derivatives of such esters, or mixtures of such esters can be utilized for this purpose. The oil can be vegetable, mineral, animal or marine in origin. Examples of non-toxic oils comprise a member selected from the group consisting of peanut oil, cottonseed oil, sesame oil, olive oil, corn oil, almond oil, mineral oil, castor oil, coconut oil, palm oil, cocoa butter, safflower, a mixture of mono- and di-glycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation is 1 wt % to 40 wt %, with the wt % of all constituents in the emulsion preparation equal to 100 wt %. The oils are disclosed in Pharmaceutical Sciences by Remington, 17^{th} Ed., pp. 403-405, (1985) published by Mark Publishing Co., in Encyclopedia of Chemistry, by Van Nostrand Reinhold, 4^{th} Ed., pp. 644-645, (1986) published by Van Nostrand Reinhold Co.; and in U.S. Pat. No. 4,259,323 issued to Ranucci.

The dosage form may contain an antioxidant to slow or effectively stop the rate of any autoxidizable material present in the dosage form, particularly if it is in the form of a gelatin capsule. Representative antioxidants comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes is about 0.001% to 25% of the total weight of the composition present in the dosage form. Antioxidants are known to the prior art in U.S. Pat. Nos. 2,707,154; 3,573,936; 3,637,772; 4,038,434; 4,186,465 and 4,559,237.

The dosage form may also contain a chelating agent to protect the active agent either during storage or when in use. Examples of chelating agents include, for example, polyacrylic acid, citric acid, edetic acid, disodium edetic acid, and the like. The chelating agent may be co-delivered with the active agent in the environment of use to preserve and protect the active agent in situ. Protection is provided for active agents which are inactivated by chelation with multivalent metal cations such as calcium, magnesium or aluminum that may be present in some foods and are at natural background levels in the fluids of the gastrointestinal tract. Such chelating agents may be combined with the self-dispersing nanoparticle active agent formulation in the porous particles, or the chelating agents may be incorporated into the drug layer in which the porous particles are dispersed.

The liquid formulation of the present invention may also comprise a surfactant or a mixture of surfactants where the surfactant is selected from the group consisting of nonionic, anionic and cationic surfactants. Exemplary nontoxic, nonionic surfactants suitable for forming a composition comprise alkylated aryl polyether alcohols known as Triton®; polyethylene glycol tertdodecyl throether available as Nonic®; fatty and amide condensate or Alrosol®; aromatic polyglycol ether condensate or Neutronyx®; fatty acid alkanolamine or Ninol® sorbitan monolaurate or Span®; polyoxyethylene sorbitan esters or Tweens®; sorbitan monolaurate polyoxyethylene or Tween 20®; sorbitan mono-oleate polyoxyethylene or Tween 80®; polyoxypropylene-polyoxyethylene or Pluronic®; polyglycolyzed glycerides such as Labraosol, polyoxyethylated castor oil such as Cremophor and polyoxypropylene-polyoxyethylene-8500 or Pluronic®. By way of example, anionic surfactants comprise sulfonic acids and the salts of sulfonated esters such as sodium lauryl sulfate, sodium sulfoethyl oleate, dioctyl sodium sulfosuccinate, cetyl sulfate sodium, myristyl sulfate sodium; sulated esters; sulfated amides; sulfated alcohols; sulfated ethers; sulfated carboxylic acids; sulfonated aromatic hydrocarbons; sulfonated ethers; and the like. The cationic surface active agents comprise cetyl pyridinium chloride; cetyl trimethyl ammonium bromide; diethylmethyl cetyl ammonium chloride; benzalkonium chloride; benzethonium chloride; primary alkylamonium salts; secondary alkylamonium salts; tertiary alkylamonium salts; quaternary alkylamonium salts; acylated polyamines; salts of heterocyclic amines; palmitoyl carnitine chloride, behentriamonium methosulfate, and the like. Generally, from 0.01 part to 1000 parts by weight of surfactant, per 100 parts of active agent is admixed with the active agent to provide the active agent formulation. Surfactants are known to the prior art in U.S. Pat. No. 2,805,977; and in 4,182,330.

The liquid formulation may comprise permeation enhancers that facilitate absorption of the active agent in the environment of use. Such enhancers may, for example, open the so-called “tight junctions” in the gastrointestinal tract or modify the effect of cellular components, such a p-glycoprotein and the like. Suitable enhancers include alkali metal salts of salicyclic acid, such as sodium salicylate, caprylic or capric acid, such as sodium caprylate or sodium caprate, and the like. Enhancers may include the bile salts, such as sodium deoxycholate. Various p-glycoprotein modulators are described in U.S. Pat. Nos. 5,112,817 and 5,643,909, which are incorporated herein by reference. Various other absorption enhancing compounds and materials are described in U.S. Pat. No. 5,824,638, which also is incorporated herein by reference. Enhancers may be used either alone or as mixtures in combination with other enhancers.

The self-dispersing nanoparticle active agent formulation of the dosage form may optionally be formulated with inorganic or organic acids or salts of drugs which promote dissolution and disintegration or swelling of the porous particles upon contact with biological fluids. The acids serve to lower the pH of the microenvironment at the porous particle, and promote rapid dissolution of a particle, such as calcium hydrogen phosphate, that is soluble in low pH environments, thus providing rapid liberation of the self-dispersing nanoparticle active agent formulation contained in the porous particle. Examples of organic acids include citric acid, tartaric acid, succinic acid, malic acid, fumaric acid and the like. Salts of drugs where the anion of the salt is acidic, such as acetate, hydrochloride, hydrobromide, sulfate, succinate, citrate, and the like, can be utilized to produce immediate disintegration and dissolution of the porous particle. A more complete list of acidic components for this application is provided in Journal of Pharmaceutical Sciences, “Pharmaceutical Salts”, Review Articles, January, (1977), Vol. 66, No. 1, pages 1-19. The interaction of an acidic component with a porous particle of, for example, calcium hydrogen phosphate, in the presence of water from gastric fluids accelerates dissolution of the particle at a greater rate than gastric fluid alone, producing a more rapid and complete release of the self-dispersing nanoparticle active agent formulation into the environment of use. Likewise alkaline components or salts of drugs where the cation of the salt is alkaline such as choline may be incorporated into the self-dispersing nanoparticle active agent formulation to promote rapid and complete dissolution of a porous particle which is soluble or swells at elevated pH. Such a particle may be formed, for example, of poly(methacrylic acid-methyl methacrylate) 1:2 available commercially as Eudragit S100 (Rohm America, Sommerset, N.J.).

In FIG. 2, a composition is illustrated which contains the porous particles 10 dispersed within a carrier 18. Typically, the composition is compacted as a tablet to form the drug layer portion of the dosage form. During the compacting phase of the manufacture, it is desired that the particle mass 11 be sufficiently non-friable so as to resist pulverization or crushing and undesired exudation of the self-dispersing nanoparticle active agent formulation.

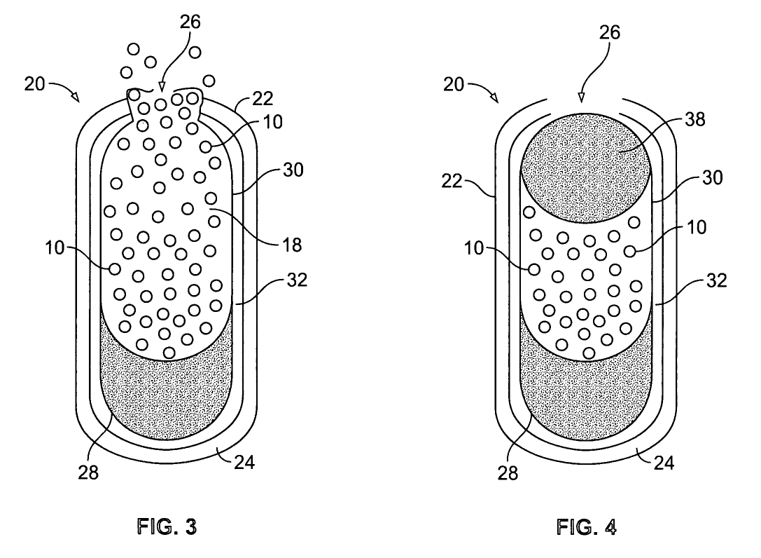

A dosage form 20 intended for continuous, zero order release of the active agent is illustrated in FIG. 3. As can be seen therein, the dosage form 20 comprises a wall 22 defining a cavity 24. Wall 22 is provided with an exit orifice 26. Within cavity 24 and remote from the exit orifice 26 is a push layer 28. A drug layer 30 is located within cavity 24 adjacent exit orifice 26. A plurality of porous particles 10 into which nanoparticles of active agent have been sorbed is dispersed in carrier 18 within the cavity 24 to form the drug layer 30. An optional, flow-promoting layer 32, the function of which will be described and which may be formed as a secondary wall, extends between drug layer 30 and the inner surface of wall 22. An orifice 26 is provided at one end of dosage form 20 to permit expression of the drug layer 30 from the dosage form upon expansion of push layer 28.

The wall 22 is formed to be permeable to the passage of an external fluid, such as water and biological fluids, and it is substantially impermeable to the passage of active agent, osmagent, osmopolymer and the like. As such, it is semipermeable. The selectively semipermeable compositions used for forming the wall are essentially nonerodible and they are insoluble in biological fluids during the life of the dosage form. Wall 22 need not be semipermeable in its entirety, but at least a portion of wall 22 should be semipermeable to allow fluid to contact or communicate with push layer 28 such that push layer 28 imbibes fluid during use. Specific materials for the fabrication of semipermeable wall 22 are well known in the art, and representative examples of such materials are described later herein.

Secondary wall 32, which functions as the flow-promoting layer or lubricant, is in contacting position with the inner surface of the semipermeable wall 22 and at least the external surface of the drug layer that is opposite wall 22; although the secondary wall 32 may, and preferably will, extend to, surround and contact the external surface of the push layer. Wall 32 typically will surround at least that portion of the external surface of the drug layer that is opposite the internal surface of wall 22. Secondary wall 32 may be formed as a coating applied over the compressed core comprising the drug layer and the push layer. The outer semipermeable wall 22 surrounds and encases the inner, secondary wall 32. Secondary wall 32 is preferably formed as a subcoat of at least the surface of the drug layer 30, and optionally the entire external surface of the compacted drug layer 30 and the push layer 28. When the semipermeable wall 22 is formed as a coat of the composite formed from the drug layer 30, the push layer 28 and the secondary wall 32, contact of the semipermeable wall 22 with the inner coat is assured.

FIG. 4 illustrates another form of the invention wherein the dosage form 20 includes a placebo layer 38 which serves to delay release of particles 10 in the environment of use. The other components of the dosage form 20 are substantially the same as those described with reference to FIG. 3, and like components are designated with the same reference numerals. The extent of the delay that may be afforded by the placebo layer will in part depend on the volume of the placebo layer 38 which has to be displaced by the push layer 28 as it imbibes fluid and expands. The placebo layer may comprise the same composition as that of the osmotic layer. The placebo layer may be formed with from just over 0 grams of composition to 400 grams depending on the delay of drug release desired. With appropriate sizing of the placebo layer, release delays of less than an hour to over eight hours as well as specific shorter periods can be achieved.

Representative polymers for forming wall 22 comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semipermeable polymers for forming the outer wall 22 comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10⁻⁵ to 10⁻² (cm. mil/atm. hr), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio.

Wall 22 also can comprise a flux regulating agent. The flux regulating agent is a compound added to assist in regulating the fluid permeability or flux through wall 22. The flux regulating agent can be a flux enhancing agent or a decreasing agent. The agent can be preselected to increase or decrease the liquid flux. Agents that produce a marked increase in permeability to fluid such as water, are often essentially hydrophilic, while those that produce a marked decrease to fluids such as water, are essentially hydrophobic. The amount of regulator in the wall when incorporated therein generally is from about 0.01% to 20% by weight or more. The flux regulator agents in one embodiment that increase flux include polyhydric alcohols, polyalkylene glycols, poilyalkylenediols, polyesters of alkylene glycols, and the like. Typical flux enhancers include polyethylene glycol 300, 400, 600, 1500, 4000, 6000 and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Representative flux decreasing agents include phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the wall 22 for imparting flexibility and elongation properties to the wall, for making wall 22 less-to-nonbrittle and to render tear strength, include phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The drug layer 30 may comprise a composition formed of a self-dispersing nanoparticle active agent formulation absorbed in porous particles, the preferred characteristics of the particles being described elsewhere herein, and a carrier 18. Depending on the release characteristics desired, the carrier may be a binder, which may be a hydrophilic polymer. The hydrophilic polymer provides a hydrophilic polymer composition in the drug layer that may contribute to the uniform release rate of active agent and controlled delivery pattern by controlling the rate of release of the porous particles containing the self-dispersing nanoparticle active agent formulation from the dosage form over a sustained period of time. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropyl methylcellulose, hydroxypropylbutylcellulose and hydroxypropylpentylcellulose; and a poly(vinylpyrrolidone) of 7,000 to 360,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

Surfactants and disintegrants may be utilized in the carrier as well. Exemplary of the surfactants are those having an HLB value of between about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like. Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

In those cases where rapid release of drug is desired, the carrier in the drug layer may be eliminated or present in only small amounts, and may comprise a binder and/or disintegrant The drug layer 30 may be formed as a mixture containing the porous particles, loaded with self-dispersing nanoparticle active agent, and the carrier. The carrier portion of the drug layer may be formed from particles by comminution that produces the desired size of the carrier particle used in the fabrication of the drug layer. The means for producing carrier particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

The active compound may be provided in the liquid active agent formulation in amounts of from 1 microgram to 5000 mg per dosage form, depending upon the required dosing level that must be maintained over the delivery period, i.e., the time between consecutive administrations of the dosage forms. More typically, loading of compound in the dosage forms will provide doses of compound to the subject ranging from 1 microgram to 2500 mg per day, more usually 1 mg to 2500 mg per day. The drug layer typically will be a substantially dry composition formed by compression of the carrier and the porous particles, with the understanding that the porous particles will have contained therein the self-dispersing nanoparticle active agent formulation. The push layer will push the drug layer from the exit orifice as the push layer imbibes fluid from the environment of use, and the exposed drug layer will be eroded to release the porous particles into the environment of use. This may be seen with reference to FIG. 3.

The push layer 28 is an expandable layer having a push-displacement composition in direct or indirect contacting layered arrangement with the drug layer 30. When in indirect contacting layered arrangement, an inert element (not shown), such as a spacer layer or disk, may be placed between the drug layer and the push layer. If several pulses of active agent are to be delivered from a single dosage form, similar inert layers may be interposed between discrete portions of drug layer. The inert layer(s) may be sized to provide appropriate time delay(s) between pulses of active agent and the volume of each discrete drug layer will provide control of the time period over which the pulse of active agent is delivered. Inert layers may be formed of materials utilized to form the push layer 28, or if desired, formed of materials that are easily compacted but do not swell in the fluid environment of use.

Push layer 28 comprises a polymer that imbibes an aqueous or biological fluid and swells to push the drug composition through the exit means of the device. Representatives of fluid-imbibing displacement polymers comprise members selected from poly(alkylene oxide) of 1 million to 15 million number-average molecular weight, as represented by poly(ethylene oxide) and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The osmagent, also known as osmotic solute and osmotically effective agent, which exhibits an osmotic pressure gradient across the outer wall and subcoat, comprises a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid raffinose, sucrose, glucose, lactose, sorbitol, inorganic salts, organic salts and carbohydrates.

Use of the inner wall or subcoat 32 is optional, but presently preferred. The inner subcoat 32 typically may be 0.01 to 5 mm thick, more typically 0.025-0.25 mm thick, although a thicker subcoat, for example 0.5 to 5 mm thick, may be used in certain applications. The inner subcoat 32 comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprises polymers having a 9,500 to 1,250,000 number-average molecular weight. For example, hydroxypropyl celluloses having number average molecular weights of between 80,000 to 850,000 are useful. The flow promoting layer may be prepared from conventional solutions or suspensions of the aforementioned materials in aqueous solvents or inert organic solvents. Prefered materials for the subcoat or flow promoting layer include hydroxypropyl cellulose, hydroxyethyl cellulose, hydroxypropyl methyl cellulose, povidone [poly(vinylpyrrolidone)], polyethylene glycol, and mixtures thereof. More prefered are mixtures of hydroxypropyl cellulose and povidone, prepared in organic solvents, particularly organic polar solvents such as lower alkanols having 1-8 carbon atoms, preferably ethanol, mixtures of hydroxyethyl cellolose and hydroxypropyl methyl cellulose prepared in aqueous solution, and mixtures of hydroxyetyyl cellulose and polyethylene glycol prepared in aqueous solution. Most preferably, the subcoat consists of a mixture of hydroxypropyl cellulose and povidone prepared in ethanol. Conveniently, the weight of the subcoat applied to the bilayer core may be correlated with the thickness of the subcoat and residual drug remaining in a dosage form in a release rate assay such as described herein. During manufacturing operations, the thickness of the subcoat may be controlled by controlling the weight of the subcoat taken up in the coating operation. When wall 32 is fabricated of a gel-forming material, contact with water in the environment of use facilitates formation of a gel or gel-like inner coat having a viscosity that may promote and enhance slippage between outer wall 22 and drug layer 30.

Exemplary solvents suitable for manufacturing the respective walls, layers, coatings and subcoatings utilized in the dosage forms of the invention comprise aqueous and inert organic solvents that do not adversely harm the materials utilized to fabricate the dosage forms. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Pan coating may be conveniently used to provide the completed dosage form, except for the exit orifice. In the pan coating system, the subcoat on the wall-forming compositions is deposited by successive spraying of the respective composition on the bilayered core comprising the drug layer and the push layer accompanied by tumbling in a rotating pan. A pan coater is used because of its availability at commercial scale. Other techniques can be used for coating the drug core. Finally, the wall or coated dosage form are dried in a forced-air oven, or in a temperature and humidity controlled oven to free the dosage form of solvent. Drying conditions will be conventionally chosen on the basis of available equipment, ambient conditions, solvents, coatings, coating thickness, and the like.

Other coating techniques can also be employed. For example, the semipermeable wall and the subcoat of the dosage form can be formed in one technique using the air-suspension procedure. This procedure consists of suspending and tumbling the bilayer core in a current of air, an inner subcoat composition and an outer semipermeable wall forming composition, until, in either operation, the subcoat and the outer wall coat is applied to the bilayer core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent. An Aeromatic® air-suspension coater can be used employing a cosolvent.

The dosage form of the invention may be manufactured by standard techniques. For example, the dosage form may be manufactured by the wet granulation technique. In the wet granulation technique a solution, suspension or dispersion of the active agent in a liquid is mixed with the porous particles to allow the self-dispersing nanoparticle active agent formulation to sorb into the pores of the porous particles. Then the carrier is blended with the porous particles using an organic solvent, such as denatured anhydrous ethanol, as the granulation fluid. After a wet blend is produced, the wet mass blend is forced through a predetermined screen onto trays. The blend is dried under ambient conditions until the desired moisture level is obtained. The drying conditions are not so severe, however, that the liquid of the self-dispersing nanoparticle active agent formulation is allowed to evaporate to any significant extent. Next, a lubricant such as magnesium stearate or agglomerated silicon dioxide (Cab-O-Sil) for example, is added to the blend, which is then put into milling jars and mixed on a jar mill for several minutes. The composition is pressed into a layer, for example, in a Manesty® press. The first compressed layer is typically the drug layer, and then the push layer may be pressed against the composition forming the drug layer, and the bilayer tablets are fed to the Kilian® Dry Coater and surrounded with the drug-free coat, followed by the exterior wall solvent coating. In those instances where a trilayer dosage form for pulsatile release having a placebo layer is to be fabicated, the placebo layer is usually formed first, then the drug layer is pressed onto the placebo layer to form a bilayer composition, and then the push layer is compressed onto the bilayer core to form the trilayer composition. The trilayer tablet is then provided with the option subcoat and the membrane coat for the rate controlling membrane. It is apparent, however, that the order in which the respective layers are compressed may be different, but the foregoing is preferred.

In another manufacture the porous particles containing the self-dispersing nanoparticle active agent formulation and other ingredients comprising the drug layer are blended and pressed into a solid layer. The layer possesses dimensions that correspond to the internal dimensions of the area the layer is to occupy in the dosage form, and it also possesses dimensions corresponding to the second layer for forming a contacting arrangement therewith. The drug layer components can also be blended with a solvent and mixed into a solid or semisolid form by conventional methods, such as ballmilling, calendering, stirring or rollmilling, and then pressed into a preselected shape. Next, the expandable layer, e.g., a layer of osmopolymer composition, is placed in contact with the layer of drug in a like manner. The layering of the drug formulation and the osmopolymer layer can be fabricated by conventional two-layer press techniques. The two contacted layers are first coated with the flow-promoting subcoat and then an outer semipermeable wall. The air-suspension and air-tumbling procedures comprise in suspending and tumbling the pressed, contacting first and second layers in a current of air containing the delayed-forming composition until the first and second layers are surrounded by the wall composition.

The dosage form of the invention is provided with at least one exit orifice. The exit orifice cooperates with the drug core for the uniform release of drug from the dosage form. The exit orifice can be provided during the manufacture of the dosage form or during drug delivery by the dosage form in a fluid environment of use. The expression “exit orifice” as used for the purpose of this invention includes a member selected from the group consisting of a passageway; an aperture; an orifice; and a bore. The expression also includes an orifice that is formed from a substance or polymer that erodes, dissolves or is leached from the outer coat or wall or inner coat to form an exit orifice. The substance or polymer may include an erodible poly(glycolic) acid or poly(lactic) acid in the outer or inner coats; a gelatinous filament; a water-removable poly(vinyl alcohol); a leachable compound, such as a fluid removable pore-former selected from the group consisting of inorganic and organic salt, oxide and carbohydrate. An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice. The exit orifice can have any shape, such as round, triangular, square, elliptical and the like for the uniform metered dose release of a drug from the dosage form. The dosage form can be constructed with one or more exits in spaced apart relation or one or more surfaces of the dosage form. The exit orifice can be performed by drilling, including mechanical and laser drilling, through the outer coat, the inner coat, or both. Exits and equipment for forming exits are disclosed in U.S. Pat. Nos. 3,845,770 and 3,916,899, by Theeuwes and Higuchi; in U.S. Pat. No. 4,063,064, by Saunders, et al.; and in U.S. Pat. No. 4,088,864, by Theeuwes, et al. The exit orifice may be from 10% to 100% of the inner diameter of the compartment formed by wall 22, preferably from 30% to 100%, and most preferably from 50% to 100%.

The continuous release dosage forms provide a uniform rate of release of compound over a prolonged period of time, typically from about zero hours, the time of administration, to about 4 hours to 20 hours or more, often for 4 hours to 16 hours, and more usually for a time period of 4 hours to 10 hours. At the end of a prolonged period of uniform release, the rate of release of drug from the dosage form may decline somewhat over a period of time, such as several hours. The dosage forms provide therapeutically effective amounts of drug for a broad range of applications and individual subject needs.

The dosage forms may also provide active agent in a pulsatile release profile. By varying the volume or weight of the placebo layer and/or the weight of the semipermeable membrane, it is possible to control the initial period before active agent is released from the dosage form. For pulse formulations, the drug layer may be formed as a rapid release layer in which the carrier in the drug layer is eliminated or is minimally present so as to allow for rapid release of the drug particles and the self-dispersing nanoparticle active agent formulation to the environment of use. The use of a disintegrant or other agent to facilitate break-up of the porous particles may be utilized. For sustained release formulations, the general considerations surrounding the selection of parameters of the push layer, the placebo layer and the semipermeable membrane to provide a desired period of delay prior to onset of delivery of the active agent will be similar as with the pulse formulation. However, as described herein, a carrier, such as a bioerodible hydrophilic polymer or the like, may generally be utilized in greater amount to provide for continuous release of the porous particles and active agent over time.

With zero order release, upon initial administration, the dosage forms may provide a drug concentration in the plasma of the subject that increases over an initial period of time, typically several hours or less, and then provide a relatively constant concentration of drug in the plasma over a prolonged period of time, typically 4 hours to 24 hours or more. The release profiles of the dosage forms of this invention provide release of drug over the entire 24-hour period corresponding to once-a-day administration, such that steady state concentration of drug in blood plasma of a subject may be maintained at therapeutically effective levels over a 24 hour period after administration the sustained release dosage form. Steady state plasma levels of drug may typically be achieved after twenty-four hours or, in some cases, several days, e.g., 2-5 days, in most subjects.

Continuous or sustained release dosage forms of this invention release drug at a uniform rate of release over a prolonged period of time as determined in a standard release rate assay such as that described herein. When administered to a subject, the dosage forms of the invention provide blood plasma levels of drug in the subject that are less variable over a prolonged period of time than those obtained with immediate release dosage forms. When the dosage forms of this invention are administered on a regular, once-a-day basis, the dosage forms of the invention provide steady state plasma levels of drug such that the difference between Cₘₐₓ and Cₘᵢₙ over the 24-hour period is substantially reduced over that obtained from administration of an immediate release product that is intended to release the same amount of drug in the 24-hour period as is provided from the dosage forms of the invention

The dosage forms of this invention may be adapted to release active agent at a uniform rate of release rate over a prolonged period of time, preferably 4-6 hours or more. Measurements of release rate are typically made in vitro, in acidified water, simulated gastric fluid or simulated intestinal fluid to provide a simulation of conditions in specific biological locations, and are made over finite, incremental time periods to provide an approximation of instantaneous release rate. Information of such in vitro release rates with respect to a particular dosage form may be used to assist in selection of dosage form that will provide desired in vivo results. Such results may be determined by present methods, such as blood plasma assays and clinical observation, utilized by practitioners for prescribing available immediate release dosage forms.

Dosage forms of the present invention having zero order release rate profiles as described herein may provide to a patient a substantially constant blood plasma concentration and a sustained therapeutic effect of active agent, after administration of the dosage form, over a prolonged period of time. The sustained release dosage forms of this invention demonstrate less variability in drug plasma concentration over a 24-hour period than do immediate release formulations, which characteristically create significant peaks in drug concentration shortly or soon after administration to the subject.

The dosage forms of the invention may have a delayed onset of action incorporated directly into the dosage form by means of the placebo layer that has been described. For particular applications, it may be desirable to deliver a plurality of the dosage forms, with or without a placebo layer or other drug layer design, at a single location in the gastrointestinal tract. This may effected conveniently by combining the dosage forms of the invention with associated technology, such as for example, the Chronset® drug delivery system of Alza Corporation, Palo Alto, Calif. Such systems can be programmed to release the dosage forms at designated times and at targeted absorption sites. That technology is described in U.S. Pat. Nos. 5,110,597; 5,223,265; 5,312,390; 5,443,459; 5,417,682; 5,498,255; 5,531,736; and 5,800,422, which are incorporated herein by reference. The composite delivery system may be manufactured by loading the osmotic dosage forms described herein into the Chronset® systems, and provide for the controlled release of active agent in a variety of formats.

An illustrative general method of manufacturing dosage forms of the invention is described below in the PREPARATION. Percentages are percentages by weight unless noted otherwise. Variations in the methods and substitution of materials may be made and will be apparent from the earlier description. Equivalent or proportional amounts of such materials may be substituted for those used in the PREPARATION below. More specific descriptions are provided in the Examples and alternative materials and procedures are illustrated therein.

### Preparation

Preparation of the Drug Layer

A binder solution is prepared by adding hydroxypropyl cellulose (Klucel MF, Aqualon Company), “HPC”, to water to form a solution containing 5 mg of HPC per 0.995 grams of water. The solution is mixed until the hydroxypropyl cellulose is dissolved. For a particular batch size, a fluid bed granulator (“FBG”) bowl is charged with the required amounts of self-dispersing nanoparticle active agent formulation and the corresponding amount of porous particles, such as exemplified by the calcium hydrogen phosphate,particles sold under the trademark FujiCalin. After the liquid is absorbed by the particles, the blend is mixed with, polyethylene oxide (MW 200,000) (Polyox® N-80, Union Carbide Corporation) (20.3%), hydroxypropyl cellulose (Klucel MF) (5%), polyoxyl 40 stearate (3%) and crospovidone (2%). After mixing the semi-dry materials in the bowl, the binder solution prepared as above is added. Then the granulation is dried in the FBG to a dough-like consistency suitable for milling, and the granulation is milled through a 7 or a 10 mesh screen.

The granulation is transferred to a tote blender or a V-blender. The required amounts of antioxidant, butylated hydroxytoluene (“BHT”) (0.01%), and lubricant, stearic acid (1%), are sized through a 40 mesh screen and both are blended into the granulation using the tote or V-blender until uniformly dispersed (about 1 minute of blending for stearic acid and about 10 minutes of blending for BHT.

Preparation of the Osmotic Push Layer Granulation

A binder solution is prepared by adding hydroxypropyl methylcellulose 2910 (“HPMC”) to water in a ratio of 5 mg of HPMC to 1 g of water. The solution is mixed until the HPMC is dissolved. Sodium chloride powder (30%) and red ferric oxide (1.0%) are milled and screened. A fluid bed granulator (“FBG”) bowl is charged with the required amounts of polyethylene oxide (MW 7,000,000) (Polyox® 303) (63.7%), HPMC (5.0%), the sodium chloride and the red ferric oxide. After mixing the dry materials in the bowl, the binder solution prepared above is added. The granulation is dried in the FBG until the target moisture content (<1% by weight water) is reached. The granulation is milled through a 7 mesh screen and transferred to a tote blender or a V-blender. The required amount of antioxidant, butylated hydroxytoluene (0.08%), is sized through a 60 mesh screen. The required amount of lubricant, stearic acid (0.25%), is sized through a 40 mesh screen and both materials are blended into the granulation using the tote or V-blender until uniformly dispersed (about 1 minute for stearic acid and about 10 minutes for BHT).

Bilayer Core Compression

A longitudinal tablet press (Korsch press) is set up with round, deep concave punches and dies. Two feed hoppers are placed on the press. The drug layer prepared as above is placed in one of the hoppers while the osmotic push layer prepared as above is placed in the remaining hopper.

The initial adjustment of the tableting parameters (drug layer) is performed to produce cores with a uniform target drug layer weight. The second layer adjustment (osmotic push layer) of the tableting parameters is performed which bonds the drug layer to the osmotic layer to produce cores with a uniform final core weight, thickness, hardness, and friability. The foregoing parameters can be adjusted by varying the fill space and/or the force setting. A typical tablet containing a target amount of drug may be approximately 0.465 inches long and approximately 0.188 inches in diameter.

Preparation of the Subcoat Solution and Subcoated System

The subcoat solution is prepared in a covered stainless steel vessel. The appropriate amounts of povidone (K29-32) (2.4%) and hydroxypropyl cellulose (MW 80,000) (Klucel EF, Aqualon Company) (5.6%) are mixed into anhydrous ethyl alcohol (92%) until the resulting solution is clear. The bilayer cores prepared above are placed into a rotating, perforated pan coating unit. The coater is started and after the coating temperature of 28-36° C. is attained, the subcoating solution prepared above is uniformly applied to the rotating tablet bed. When a sufficient amount of solution has been applied to provide the desired subcoat weight gain, the subcoat process is stopped. The desired subcoat weight will be selected to provide acceptable residuals of drug remaining in the dosage form as determined in the release rate assay for a 24-hour period. Generally, it is desirable to have less than 10%, more preferably less than 5%, and most preferably less than 3% of residual drug remaining after 24 hours of testing in a standard release rate assay as described herein, based on the initial drug loading. This may be determined from the correlation between subcoat weight and the residual drug for a number of dosage forms having the same bilayer core but different subcoat weights in the standard release rate assay.

Preparation of the Rate Controlling Membrane and Membrane Coated System

Subcoated bilayer cores prepared as above are placed into a rotating, perforated pan coating unit. The coater is started, and after the coating temperature (28-38° C.) is attained, a coating solution such as illustrated in A, B or C below is uniformly applied to the rotating tablet bed until the desired membrane weight gain is obtained. At regular intervals throughout the coating process, the weight gain is determined and sample membrane coated units may be tested in the release rate assay to determine a T₉₀ for the coated units. Weight gain may be correlated with T₉₀ for membranes of varying thickness in the release rate assay. When sufficient amount of solution has been applied, conveniently determined by attainment of the desired membrane weight gain for a desired T₉₀, the membrane coating process is stopped.

Illustrative Rate Controlling Membrane Compositions:

A coating solution is prepared in a covered stainless steel vessel. The appropriate amounts of acetone (5650 g) and water (297 g) are mixed with the poloxamer 188 (16 g) and cellulose acetate (297 g) until the solids are completely dissolved. The coating solution has about 5% solids upon application.

Acetone (5054 g) is mixed with cellulose acetate (277.2 g) until the cellulose acetate is completely dissolved. Polyethylene glycol 3350 (2.8 g) and water (266 g) are mixed in separate container. The two solutions are mixed together until the resulting solution is clear. The coating solution has about 5% solids upon application.

Acetone (7762 g) is mixed with cellulose acetate (425.7 g) until the cellulose acetate is completely dissolved. Polyethylene glycol 3350 (4.3 g) and water (409 g) are mixed in separate container. The two solutions are mixed together until the resulting solution is clear. The coating solution has about 5% solids upon application.

Drilling of Membrane Coated Systems

One exit port is drilled into the drug layer end of the membrane coated system. During the drilling process, samples are checked at regular intervals for orifice size, location, and number of exit ports.

Drying of Drilled Coated Systems

Drilled coated systems prepared as above are placed on perforated oven trays which are placed on a rack in a relative humidity oven at 40° C. (43-45% relative humidity) and dried to remove the remaining solvents from the coating layers.

Color and Clear Overcoats

Optional color or clear coats solutions are prepared in a covered stainless steel vessel. For the color coat 88 parts of purified water is mixed with 12 parts of Opadry II [color not critical] until the solution is homogeneous. For the clear coat 90 parts of purified water is mixed with 10 parts of Opadry Clear until the solution is homogeneous. The dried cores prepared as above are placed into a rotating, perforated pan coating unit. The coater is started and after the coating temperature is attained (35-45° C.), the color coat solution is uniformly applied to the rotating tablet bed. When sufficient amount of the dispersion has been applied, as conveniently determined when the desired color overcoat weight gain has been achieved, the color coat process is stopped. Next, the clear coat solution is uniformly applied to the rotating tablet bed. When sufficient amount of solution has been applied, or the desired clear coat weight gain has been achieved, the clear coat process is stopped. A flow agent (e.g., Car-nu-bo wax) is applied to the tablet bed after clear coat application.

Variations in the foregoing procedure will be apparent to one skilled in the art. The examples are provided to illustrate representative dosage forms of the invention prepared by analogous methods.

### Assay

The release rate of drug from devices containing the dosage forms of the invention may be determined in standardized assays such as the following. The method involves releasing systems into a release liquid medium, such as acidified water (pH 3), artificial gastric fluid or artificial intestinal fluid. Aliquots of sample release rate solutions are injected onto a chromatographic system to quantify the amount of drug released during specified test intervals. Drug is resolved on a C₁₈ column and detected by UV absorption at the appropriate wavelength for the drug in question. Quantitation is performed by linear regression analysis of peak areas from a standard curve containing at least five standard points.

Samples are prepared with the use of a USP Type 7 Interval Release Apparatus. Each system (invention device) to be tested is weighed. Then, each system is glued to a plastic rod having a sharpened end, and each rod is attached to a release rate dipper arm. Each release rate dipper arm is affixed to an up/down reciprocating shaker (USP Type 7 Interval Release Apparatus), operating at an amplitude of about 3 cm and 2 to 4 seconds per cycle. The rod ends with the attached systems are continually immersed in 50 ml calibrated test tubes containing 50 ml of the release medium, equilibrated in a constant temperature water bath controlled at 37° C.±0.5° C. At the end of each time interval specified, typically one hour or two hours, the systems are transferred to the next row of test tubes containing fresh release medium. The process is repeated for the desired number of intervals until release is complete. Then the solution tubes containing released drug are removed and allowed to cool to room temperature. After cooling, each tube is filled to the 50 ml mark, each of the solutions is mixed thoroughly, and then transferred to sample vials for analysis by high pressure liquid chromatography (“HPLC”). A standard concentration curve is constructed using linear regression analysis. Samples of drug obtained from the release test are analyzed by HPLC and concentration of drug is determined by linear regression analysis. The amount of drug released in each release interval is calculated. Alternatively, concentration of drug may be determined by uv analysis.

Examples 1 and 2, below, illustrate the greater drug loading possible by using nanoparticles of active agent in a drug form having enhanced dissolution characterics. In each example, the same active agent is used and the porous particle carrier, loaded with liquid carrier, performs and can be handled as fine dry granules. In Example 1, the active agent is dissolved into the liquid carrier to its maximum soluble concentration. In Example 2, nanoparticles of the active agent are produced, suspended in the liquid carrier and then loaded into the porous carrier.

### EXAMPLE 1

A dosage form such as is illustrated in FIG. 3, having a total drug layer weight of 500 mg, is formed comprising an active agent that is dissolved in a liquid carrier, a liquid carrier, a porous carrier and other dosage form materials as set out below. In this hypothetical example, the active agent is at its maximum concentration in the liquid carrier at 20 mg of the drug per gram of the liquid carrier.

| | | |
|---|---|---|
| active agent (solubilized in liquid carrier) | 4.4 | mg |
| liquid carrier | 222.8 | mg |
| porous carrier | 222.8 | mg |
| other materials | 50 | mg |
| Total | 500 | mg |

### EXAMPLE 2

A dosage form such as is illustrated in FIG. 3, having a total weight of 500 mg, is formed comprising an active agent that is dispersed in a liquid carrier, a liquid carrier, a porous carrier and other dosage form materials (including a push layer) as set out below. The active agent is in the form of nanoparticles, suspended in the liquid carrier and then loaded into the porous carrier.

| | | |
|---|---|---|
| active agent (solubilized in liquid carrier) | 3.6 | mg |
| active agent (in nanoparticle form) | 84.4 | mg |
| liquid carrier | 181.0 | mg |
| porous carrier | 181.0 | mg |
| other materials | 50.0 | mg |
| Total | 500 | mg |

As can be seen in Example 2, by including the active agent in the drug form as nanoparticles in the liquid carrier, a twenty-fold increase in drug loading in the dosage form is obtained over the dosage form of Example 1. Importantly, also, the dosage form of Example 2, because of the effect of the self-dispersing liquid carrier and the dissolution characteristics of the nano sized particles, still maintains high dissolution characteristics.

Additionally, with self-dispersing nanoparticle formulations loaded into the porous carrier according to the present invention, the self-dispersing nanoparticle formulations can be handled as fine dry particles in the production of dosage forms. The loaded porous carrier can be used to produce solid dosage forms, and, indeed, solid dosage forms that have high drug loading, high dissolution characteristics and high drug bioavailability.

### EXAMPLE 3

Nanoparticles of megestrol acetate were prepared by making an aqueous suspension of megestrol acetate in 2% Pluronic F108. The suspension was milled for 4 hours on the Dynomill, producing a mean particle size of 0.3 micron. To stabilize the milled drug a polymer solution of hydroxypropyl methylcellulose (HPMC E5) was added to a ratio of Pluronic F108:HPMC E5 1:2. The final milled suspension was then freeze-dried and the resulting nanoparticles had a concentration of 71.2% megestrol acetate.

134 mg of the freeze-dried nanoparticles of megestrol acetate were dispersed into 480 mg of the self-emulsifying liquid carrier (Capric Acid/Cremophor EL, 50/50) and mixed well to get a suspension of nanoparticles. To convert the suspension into a solid form, 888 mg of Neusilin granules were gradually added into the suspension and mixed well. The final Neusilin/suspension blend produced fine, dry granules. Other excipients, 16 mg Magnesium Stearate and 24 mg Cross Carmellose Sodium (Ac-di-sil), were added to the granules and mixed well. Then, the granules were passed through a 40-mesh screen and tumbled for 30 minutes for further mixing. Finally, the powder was tabletted on a Carver Press with ¼″ standard concave tooling. The final 20 mg megestrol acetate tablet weighed 309 mg and had a final composition as listed in the Table 1.

TABLE 1

| Component | Wt % | Mg per dose |
|---|---|---|
| Megestrol | 6.47% | 20.0 |
| Plurnoic F108 | 0.75% | 2.3 |
| HPMC E5 | 1.49% | 4.6 |
| Neusilin | 57.58% | 178.0 |
| Capric Acid | 15.57% | 48.1 |
| Cremophor EL | 15.57% | 48.1 |
| Mg St | 1.03% | 3.2 |
| Acdisol | 1.54% | 4.8 |

### EXAMPLE 4

The procedure of Example 3 was repeated in this example for providing the following dosage form:

A dosage form, the amount of each component added was identical to that of Example 3, except that the amount of Acdisol added was ⅓ that in Example 3. The final 20 mg megestrol acetate tablet weighed 305 mg.

### EXAMPLE 5

The procedure of Example 3 was repeated in this example for providing the following dosage form:

A dosage form, the amount of each component added was identical to that of Example 3, except that the amount of Acdisol added was ⅔ of that in Example 3. The final 20 mg megestrol acetate tablet weighed 307 mg.

### EXAMPLE 6

The procedure of Example 3 was repeated in this example for providing the following dosage form:

A dosage form, the amount of each component added was identical to that of Example 3, except that the amount of Acdisol added was 2 times that of Example 3. The final 20 mg megestrol acetate tablet weighed 313 mg.

### EXAMPLE 7

The procedure of Example 3 was repeated in this example for providing the following dosage form:

A dosage form, the amount of each component added was identical to that of Example 3, except that the amount of Acdisol added was 3 times that of Example 3. The final 20 mg megestrol acetate tablet weighed 318 mg.

Nanoparticles of drugs for use according to embodiments of the present invention can be prepared using any process providing particles within a desired range of sizes. For example, the drug may be processed using a wetmilling or supercritical fluid process, such as an RESS or GAS process. In addition, processes for producing nanoparticles are disclosed in U.S. Pat. Nos. 6,267,989, 5,510,118, 5,494,683, and 5,145,684. Nanoparticles may also be formed according to methods described elsewhere herein for the formation of drug particles.

In the use of nanoparticles according to some embodiments of the present invention, it is useful to process the drug or nanoparticles of drug with one or more coating agents to minimize particle aggregation or agglomeration. Exemplary coating agents include lipids, hydrophilic polymers, such as hydroxypropyl methylcellulose (“HPMC”) and polyvinylpyrrolidone (“PVP”) polymers, and solid or liquid surfactants. The coating agent used in a nanoparticle forming process may also include a mixture of agents, such as a mixture of two different surfactants. Where used as a coating agent, a hydrophilic polymer may work to both facilitate formation of nanoparticulate material and stabilized the resulting nanoparticles against recrystalization over long periods of storage. Surfactants useful as coating agents in the creation of nanoparticles useful in the self-emulsifying nanosuspension of the present invention include nonionic surfactants, such as Pluronic F68, F108, or F127. the non-ionic surfactants already mentioned herein may also be useful as coating agents in a nanoparticle forming process.

In-vitro and in-vivo studies were conducted using the solid dosage forms of Example 3. Example 8 describes the in-vitro study to determine drug release profiles and Example 9 describes the in-vivo study to determine drug bioavailability.

### EXAMPLE 8

The release profile of megestrol acetate from the solid dosage form of Example 3 in artificial intestinal fluid (“AIF”) was conducted in a USP apparatus 11. The release medium was 500 ml of AIF with 2% Pluronic F108. The paddle agitation speed was 100 rpm. The concentration of megestrol acetate was assayed using a UV-spectrophotometer at 290 nm of wavelength.

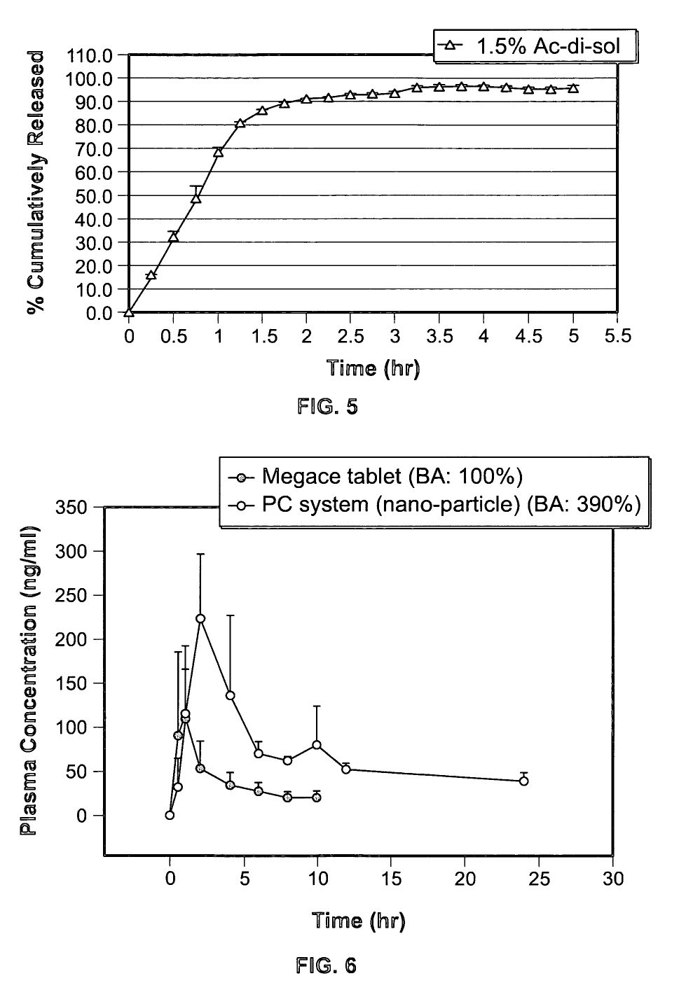

FIG. 5 illustrates the release profile (cumulative release of drug as a function of time measured from immersion of the drug forms in AIF) of megestrol acetate as measured in Example 8.

### EXAMPLE 9

A two-arm PK study was conducted with 3 fasted mongrel dogs. The two arms were, respectively, an immediate release (IR) Megace® tablets (20 mg) and a tablet prepared according to Example 3 herein. The drug dose was 20 mg for both arms. Plasma samples for the Megace tablets were taken at 1, 2, 4, 6, 8 and 10 hours after dosing of the IR dosage form. Plasma samples for the dosage form prepared according to Example 3 were taken at 0, 1, 2, 4, 6, 8, 10, 12 and 24 hours after dosing. The plasma samples were measured using a LC/MS method with minimum detection limit of 1 ng/ml.

FIG. 6 illustrates the bioavailability of megestrol acetate as determined in Example 9. The plasma concentration of megestrol acetate in ng/ml is plotted versus time in hours. The error bars represent the standard deviation of n=3.

Table 2 shows analysis of the results of Example 9 and relates to the data illustrated in FIG. 6. For the data presented in Table 2, AUC_{inf} was calculated by adding AUCt and AUC_{t-inf}, where AUCt was estimated by trapezoidal integration to the last sampling point (t) and AUC_{t-inf} was estimated by integration from t to infinity. BA % is relative to that of the Megace tablet. Megestrol acetate plasma level was measured with an LC/MS method.

As shown in Table 2, the bioavailability of megestrol acetate from the dosage form of Example 11 was 3.9 times that of the Megace IR tablet.

TABLE 2

| | Cₘₐₓ, sd | CV of | Tₘₐₓ, sd | AUC_{inf}, sd | CV of AUC | BA |
|---|---|---|---|---|---|---|
| | (ng/mL) | Cₘₐₓ (%) | (h) | (Ng*h/mL) | (%) | (%) |
| Megace tablet | 113, 79 | 70.1 | 0.8, 0.3 | 506, 251 | 50 | 100 |
| Tablet of Example | 223, 74 | 33 | 2, 0 | 2219, 443 | 20 | 390 |
| 11 | | | | | | |

The present invention is described and characterized by one or more of the following technical features and/or characteristics, either alone or in combination with one or more of the other features and characteristics: a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation, the dosage form optionally having a placebo layer between the exit orifice and the drug layer; a dosage form comprising a flow-promoting layer interposed between the inner surface of the wall and at least the external surface of the drug layer located within the cavity; a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦0.5 or 0≦m≦2.0, the dosage form optionally having a placebo layer between exit orifice and the drug layer; a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a specific volume of at least 1.5 ml/g, a BET specific surface area of at least 20 m²/g, and a water absorption capacity of at least 0.7 ml/g, the dosage form optionally having a placebo layer between the exit orifice and the drug layer; a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh, the dosage form optionally having a placebo layer between the exit orifice and the drug layer; a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a bulk specific volume of 1.5 ml/g-5 ml/g, a BET specific area of 20 m²/g-60 m²/g, a water absorption capacity of at least 0.7 ml/g, and a mean particle size of 50 microns or greater, the dosage form optionally having a placebo layer between the exit orifice and the drug layer; a dosage form for an active agent comprising a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation, the porous particles being formed from material selected from calcium hydrogen phosphate, magnesium aluminometasilicates, microcrystalline celluloses and silicon dioxides; a dosage form comprising at least two drug layers separated by at least one inert layer; a dosage form comprising at least two drug layers, each of said drug layers containing a different active agent; a method of facilitating the release of an active agent from a dosage form comprising sorbing a liquid formulation of the active agent into a plurality of porous particles, the particles, having a mean particle size of 5-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
   CaHPO₄.mH₂O
wherein m satisfies the relationship 0≦m≦0.5 or 0≦m≦2.0, and dispersing the particles throughout a bioerodible carrier; a composition comprising a liquid formulation of an active agent sorbed into a plurality of porous particles, the particles being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
   CaHPO₄.mH₂O
wherein m satisfies the relationship 0≦m≦0.5 or 0≦m≦2.0, and dispersed throughout a bioerodible carrier, the particles being released in the environment of use over a prolonged period of time; a dosage form wherein the self-dispersing nanoparticle active agent formulation comprises a self-emulsifying formulation; a dosage form wherein the active agent has low water solubility; a dosage form wherein the self-dispersing nanoparticle active agent formulation comprises an absorption enhancer; a dosage form wherein the self-dispersing nanoparticle active agent formulation comprises at least 30% by weight of the drug layer; dosage form wherein the porous particle comprises magnesium aluminometasilicate represented by the general formula
   Al₂O₃MgO.2SiO₂.nH₂O
wherein n satisfies the relationship 0≦n≦10; a dosage form wherein the porous particle comprises magnesium aluminometasilicate represented by the general formula
   Al₂O₃MgO.2SiO₂.nH₂O
wherein n satisfies the relationship 0≦n≦10 and having a specific surface area of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g; a dosage form having placebo layer located between the drug layer and an exit orifice; a dosage form comprising a pH regulating agent selected from organic acids, inorganic acids and bases; a dosage form comprising a chelating agent.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined herein.

What is claimed is:

1. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer;
the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation.

2. The dosage form of claim 1, wherein a flow-promoting layer is interposed between the inner surface of the wall and at least the external surface of the drug layer located within the cavity.

3. The dosage form of claim 1, wherein a placebo layer to delay onset of delivery of the active agent optionally is placed between the drug layer and the exit orifice.

4. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles; the porous particles having a mean particle size of ranging from about 50 to about 150 microns and being formed by spray drying a scale-like calcium hydrogen phosphate with a specific surface area of about 20 m²/g to about 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0.

5. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a specific volume of at least 1.5 ml/g, a BET specific surface area of at least 20 m²/g, and a water absorption capacity of at least 0.7 ml/g.

6. The dosage form of claim 5, wherein the porous particles have a bulk density of 0.4-0.6 g/ml, a BET surface area of 30-50 m²/g, a specific volume of greater than 2 ml/g, and a mean pore size of at least 50 Angstroms.

7. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh.

8. The dosage form of claim 7, wherein the particles have a size distribution of 100% is less than 40 mesh, 60%-90% is less than 100 mesh and 20%-60% is less than 200 mesh.

9. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being calcium hydrogen phosphate having a bulk specific volume of 1.5 ml/g-5 ml/g, a BET specific area of 20 m²/g-60 m²/g, a water absorption capacity of at least 0.7 ml/g, and a mean particle size of at least 70 micrometers.

10. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation.

11. The dosage form of claim 10, wherein the dosage form comprises a placebo layer between the exit orifice and the drug layer.

12. The dosage form of claim 10, wherein a flow-promoting layer is interposed between an inner surface of the wall and at least an external surface of the drug layer located within the cavity.

13. A method of facilitating the release of an active agent from a dosage form comprising
sorbing a self-dispersing nanoparticle active agent formulation of the active agent into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0; and
dispersing the particles throughout a bioerodible carrier.

14. A composition comprising a self-dispersing nanoparticle active agent formulation of the active agent sorbed into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an verage particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0, and dispersed throughout a bioerodible carrier, the particles being released in the environment of use over a prolonged period of time.

15. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being magnesium aluminometasilicate.

16. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in porous particles, the porous particles being magnesium aluminometasilicate represented by the general formula
Al₂O₃MgO.2SiO₂.nH₂O
wherein n satisfies the relationship 0≦n≦10.

17. A dosage form for an active agent comprising
a wall defining a cavity, the wall having an exit orifice formed or formable therein and at least a portion of the wall being semipermeable;
an expandable layer located within the cavity remote from the exit orifice and in fluid communication with the semipermeable portion of the wall;
a drug layer located within the cavity adjacent the exit orifice and in direct or indirect contacting relationship with the expandable layer; the drug layer comprising a self-dispersing nanoparticle active agent formulation absorbed in and/or onto porous particles, the porous particles being magnesium aluminometasilicate represented by the general formula
Al₂O₃MgO.2SiO₂.nH₂O
wherein n satisfies the relationship 0≦n≦10 and having a specific surface area of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g.

18. A composition of nanoparticles of an active agent suspended in a liquid carrier and sorbed into porous particle carriers.

19. A dosage form comprising a self-dispersing nanoparticle formulation loaded into one or more porous carriers and wherein the nanoparticles have a mean particle size less than 2000 nm.
